(19) United States
(12) Patent Application Publication
(10) Pub. No.: US 2008/0015374 A1
(43) Pub. Date: Jan. 17, 2008

(54) METHOD FOR THE SYNTHESIS AND ISOLATION OF PHYTOSTEROL ESTERS

(75) Inventors: David B. Wiley, Coshocton, OH (US); Thomas A. Dobbins, Howard, OH (US)

Correspondence Address: THOMPSON HINE L.L.P.;Intellectual Property Group, P.O. BOX 8801, DAYTON, OH, 45401-8801, US

(73) Assignee: WILEY ORGANICS, INC., Coshocton, OH (US)

(21) Appl. No.: 11/776,181

(22) Filed: Jul. 11, 2007

Related U.S. Application Data
(60) Provisional application No. 60/819,847, filed on Jul. 11, 2006.

(51) Int. Cl. C07J 9/00

(52) U.S. Cl. 552/544

(57) ABSTRACT

A process for the synthesis and isolation of phytosterol esters is described, wherein an alkali metal borohydride is used to reductively bleach tocoquinones and other chromophores in phytosterol ester trans-esterification reaction mixtures. Phytosterol esters produced by this method possess the traditional hallmarks of quality, namely bland taste and light color, and are suitable for food and pharmaceutical applications.

### CROSS REFERENCE TO RELATED APPLICATION

This application claims the benefit of U.S. Provisional Application No. 60/819,847, filed Jul. 11, 2006, the contents of which are hereby incorporated by reference.

### BACKGROUND OF THE INVENTION

Plant sterols or “phytosterols” are abundant in nature, occurring naturally in a variety of fruits and vegetables that are part of the human diet. These compounds cannot be manufactured by the human body and are obtained exclusively through the diet. In the United States, the average intake of phytosterols is approximately 250 milligrams per day; vegetarians consume nearly twice as much. Because phytosterols structurally resemble cholesterol, they compete with cholesterol for absorption in the gastrointestinal tract, providing substantial health benefits by helping to maintain healthy levels of blood serum cholesterol. Long term clinical studies and studies with dose levels of up to 25,000 mg phytosterols per day (i.e. 100 times the average dietary intake) have been conducted. Over 1,800 men, women, adolescents, and children have participated in these studies, some of which date back to the 1950's. To date, no significant adverse health effects have been reported.

Fatty acid esters of sterols and/or stanols are currently employed to replace a substantial portion of the undesirable saturated and trans-unsaturated fats in foods such as margarines, mayonnaise, cooking oils, cheeses, butter and shortening. Recently marketed vegetable spreads such as Benecol® and Take Control® are two examples of phytosterol-containing products intended to promote healthy cholesterol levels. These vegetable oil spreads contain up to 20% by weight added phytosterols in the form of fatty acid esters. Because of the similarity of the physical properties of these esters to those of undesirable hardstock fats, the substitution or replacement contributes favorably to the flavor, texture and other sensory properties of the foods. Only the fatty acid portion of the phytosterol esters is digested or absorbed; the sterol portion is not absorbable, thereby reducing total caloric uptake.

The phytosterol esters defined herein comprise unsaturated and saturated fatty acid esters of sterols or stanols as well as mixtures thereof. The term phytosterol is intended to mean saturated and unsaturated sterol alcohols and their blends derived from botanical sources (oilseeds or conifers), as well as synthetically produced sterol alcohols and their blends having properties that replicate those of naturally occurring alcohols. These sterol alcohols are characterized by a common polycyclic steroid nucleus comprising a 17-carbon atom ring system, a side chain, and a hydroxyl group. The nucleus is either saturated, wherein the sterol alcohol is referred to as a stanol, or unsaturated, wherein the sterol alcohol is referred to as a sterol. For purposes of the present invention, sterol is understood to mean a single sterol or blends of sterols, and stanol is understood to mean a single stanol or blends of stanols.

Early attempts to synthesize the fatty acid esters of phytosterols involved reacting sterols with free fatty acids or the anhydrides of free fatty acids. For example, German Patent Number 2035069, which relates to the addition of sterol fatty acid esters to cooking oils with the objective of lowering blood serum cholesterol levels, describes the synthesis of phytoserol esters by reacting free sterols with fatty acid anhydrides using toxic catalysts like perchloric acid or thionyl chloride. This synthetic method is inconsistent with the requirements for the preparation of a food-grade product, since both the fatty acid anhydride reagent and the specified catalysts are unacceptable in a food process.

Japanese Patent 76-11113 discloses a catalyst-free process for producing fatty acid esters of phytosterols and related compounds. This process employs a significant molar excess of fatty acid (a minimum of 25% up to 50%), which in turn requires the use of a costly, laborious alkali refining process to recover the phytosterol ester product. Moreover, the isolation techniques disclosed result in a phytosterol ester product that is dark in color and has poor organoleptic properties.

U.S. Pat. Nos. 5,892,068 and 6,410,758 teach methods of synthesizing phytosterol esters by reacting a sterol or stanol or mixtures thereof with a free fatty acid in the presence of a very modest amount (less than 0.15% by weight) of far less toxic acidic catalysts like toluenesulfonic acid, methanesulfonic acid, sodium hydrogen phosphate, or sodium bisulfite and a “color deactivating agent” like carbon, charcoal, carbon black, bleaching earth (i.e. Bentonite or Fuller's Earth), or silica. However, the acidic catalyst remains in the final product, which is not subjected to washing to effect catalyst removal. The presence of residual catalyst is undesirable in a food-grade or pharmaceutical-grade product. The contents of these patents are hereby incorporated by reference.

An alternate synthetic method involving the transesterification reaction of sterols with the fatty acid esters of low molecular weight alcohols (typically methanol or ethanol) under alkaline conditions has proven to yield a superior phytosterol ester product than reactions between sterols and free fatty acids or fatty acid anhydrides. The transesterification method of synthesizing sterol esters has been widely adopted by the food industry.

German Patent Number 2248921 describes an improved method for esterifying the sterols present in oils and fats by a transesterification technique that fulfills the criteria of a food-grade process. Free sterol and a stoichiometric excess of fatty acid esters of a low molecular weight aliphatic alcohol are added to a mixture of oil or fat. The resulting mixture is transesterified by means of an alkali metal alkoxide catalyst at elevated temperature and reduced pressure. The methanol produced by this reaction is volatilized and removed by distillation as the reaction proceeds. The contents of this patent are hereby incorporated by reference.

U.S. Pat. No. 5,502,045 disclosed the transesterification of stanols with a fatty acid ester from an edible oil to produce a waxy sterol ester mixture with improved fat solubility characteristics. Specifically, this patent describes the reaction of sitostanol transesterified with the methyl esters of the fatty acids that occur in edible oils such as rapeseed oil, specifically via a base-catalyzed trans-esterification reaction. The contents of this patent are hereby incorporated by reference.

U.S. Pat. No. 6,231,915 describes a “solvent-free” process for synthesizing phytosterol esters by reacting the methyl esters of fatty acids using an alkali metal alkoxide such as “sodium methylate” (sodium methoxide). The contents of this patent are hereby incorporated by reference.

U.S. Pat. No. 6,162,483 also describes a process for synthesizing phytosterol esters that relies on the transesterification process that is now widely employed by the edible fat and oil industry, i.e., reacting a free sterol or stanol with a fatty acid ester or mixture of fatty acid esters in the presence of a catalytic amount of an alkali metal alkoxide catalyst. The fatty acid ester is used in excess and functions as a solvent, solubilizing the sterol or stanol under the conditions employed (elevated temperatures of 90° C. to 120° C. at reduced pressures of 5 to 15 mm Hg). The reaction yields a mixture of phytosterol esters and unreacted free fatty acid esters. The phytosterol esters are concentrated by vacuum distillation, which removes some but not all of the excess of fatty acid esters. The contents of this patent are hereby incorporated by reference.

U.S. Pat. Nos. 6,174,560, 6,184,397, and 6,441,206 also teach the synthesis of phytosterol esters by transesterification of free stannols or sterols with fatty acid methyl esters. The disclosures of these patents are incorporated herein by reference.

Although transesterification quickly emerged as the preferred synthetic method and has been widely employed for many years, both the color and the organoleptic properties of the phytosterol ester products often remain problematic. After the transesterification reaction is complete, activated carbon, silica, or “bleaching clays” like Bentonite or Fuller's Earth are usually employed to reduce the color of the product. In addition, the soaps produced by the alkali metal alkoxide catalysts must be removed, as well as the excess fatty acid methyl esters.

U.S. Pat. No. 6,184,397 describes a two-step organic solvent/acidic aqueous solvent isolation technique. The phytosterol ester is extracted into an organic solvent and subsequently isolated after evaporation. Typical organic solvents include dichloromethane, chloroform or toluene. None of these solvents is suitable for a food-grade or pharmaceutical-grade product, but the inventors note that when light, non-chlorinated hydrocarbon solvents like hexane are employed, the formation of inseparable emulsions is observed. The solution of phytosterol esters in the organic solvent is then washed several times with an aqueous solution of sodium bisulfite or other acidic reagent. The fatty acid soaps are partitioned into the aqueous phase and removed. The remaining organic phase containing the isolated ester is then dried over anhydrous sodium sulfate and decolorized with activated charcoal. The purified phytosterol esters are recovered by removing the organic solvent using a rotary evaporator. However, the level of fatty acid methyl esters is not appreciably reduced by this work-up procedure.

U.S. Pat. No. 6,184,397 describes a “preferred isolation technique” in which the crude phytosterol ester post-reaction mixture is washed with one-tenth its volume of water. The immiscible phases are then allowed to separate for one to two hours, then the aqueous layer is removed and discarded. The removal of soaps by this method is inefficient, however, and removal of fatty acid methyl esters does not occur. The resulting washed phytosterol ester is then decolorized using a bleaching clay or a silica-based bleaching aid to remove color (and allegedly a portion of the soaps), then “steam deodorized” to remove excess fatty acid methyl esters, which are then recycled without further processing.

### SUMMARY OF THE INVENTION

The present invention relates to a method for the synthesis of phytosterol esters from a mixture containing free sterols and fatty acid esters of low molecular weight aliphatic alcohols by contacting the mixture with a reductive bleaching agent and reacting the sterols and fatty acid esters in the presence of sufficient catalyst to produce the corresponding phytosterol esters. Phytosterol esters produced by the method described herein exhibit the desired qualities of bland taste and light color, and are suitable for food and pharmaceutical applications.

In accordance with a particular aspect of the present invention, a process for the synthesis and isolation of phytosterol esters is described, wherein an alkali metal borohydride is used to reductively bleach tocoquinones and other chromophores in phytosterol ester transesterification reaction mixtures.

### DETAILED DESCRIPTION OF THE INVENTION

The present invention relates to methods for removing or reducing the undesirable pigments present in phytosterol esters or their precursors. The disclosed method can be used as an alternative to the post-reaction decolorization using activated carbon, silica, or bleaching clays, or as a means of augmenting the efficiency of post-reaction color removal. Commercial sterols isolated from botanical sources, especially oilseeds, contain traces of phenolic compounds, notably tocopherols. These pale yellow viscous oils are very readily oxidized to form tocoquinones. Traces of such unsaturated compounds may also be found, albeit usually to a lesser degree, in some of the commercial fatty acid methyl esters employed as reactants in the synthesis of phytosterol esters by transesterification.

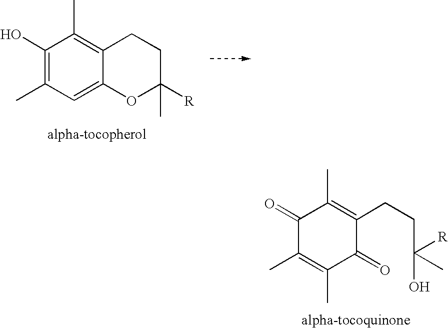

Tocoquinones are intense pigments that are a major source of the undesirable color of many phytosterol ester products produced by transesterification. The intensity of the color exhibited by these compounds is further enhanced under the alkaline conditions that prevail when an alkali metal alkoxide catalyst is employed.

To reduce or eliminate these intensely colored oxidation products, a reductive bleaching agent may be employed before or during the transesterification synthesis reaction. Such a reductive bleaching agent can be employed in conjunction with the alkali metal alkoxide catalyst or it may alternatively serve as a catalyst in its own right.

Any reductive bleaching agent employed in this fashion should not only promote or at least not inhibit the transesterification reaction, it should also be inert to the reactants (sterols and fatty acid esters) and to the products (sterol esters). Some alkali metal borohydrides, notably sodium borohydride and potassium borohydride, satisfy these criteria.

Sodium borohydride, also known as sodium tetrahydridoborate, has the chemical formula NaBH₄. It is a highly selective reducing agent widely employed in the manufacture of pharmaceuticals, intermediates, and fine chemicals. Pure sodium borohydride is a white crystalline solid that in the absence of moisture is stable up to approximately 400° C. Insoluble in hydrocarbons and ether, it is soluble in cold water but decomposes in hot water, evolving hydrogen and making sodium borate.

In contrast to lithium aluminum hydride, sodium borohydride is insoluble in hydrocarbons and ethereal solvents, but is soluble in methanol, ethanol, isopropanol, and other water-miscible aliphatic alcohols. Sodium and potassium borohydrides are much milder reducing agents than lithium borohydride. In hydroxylic solvents they rapidly reduce aldehydes and ketones to alcohols even at 25° C., but they are essentially inert to other functional groups such as esters, lactones, and carboxylic acids.

While sodium borohydride is quite soluble in methanol, forming a 14% by weight solution at 20° C., it reacts with methanol at an appreciable rate. Consequently, although methanol can serve as a convenient solvent for the delivery and rapid dispersal of sodium borohydride to phytosterol ester reaction mixtures, methanoic solutions of sodium borohydride must be freshly prepared.

Sodium borohydride is also supplied commercially in a stabilized aqueous solution with a typical composition of 48% water, 40% sodium hydroxide, and 12% sodium borohydride. This solution is stable and has an indefinitely long shelf life. Furthermore, it can be further diluted with water or with alcohols such as methanol. Most aldehydes are reduced so rapidly by this solution that any condensation reactions promoted by the sodium hydroxide do not interfere. This aqueous solution of sodium borohydride and sodium hydroxide is the most convenient method for handling and delivering sodium borohydride.

Sodium borohydride is also environmentally benign. The oxidation product of sodium borohydride is sodium borate, a comparatively inert, non-toxic, water-soluble compound that can be readily removed from the reaction mixture by washing with water, or, preferably, with a dilute aqueous solution of a weak volatile organic acid such as acetic acid.

When sodium borohydride is employed in conjunction with a conventional alkoxide transesterification catalyst like sodium methoxide, the resulting phytosterol ester is generally 2 to 5 points lighter on the Gardner Scale than when sodium methoxide alone is employed. The Gardner Scale is a visual scale (described in ASTM D1544, “Standard Test Method for Color of Transparent Liquids”) originally developed during the 1920s to describe the color of drying oils, varnishes, fatty acids, polymerized fatty acids and resin solutions. These liquids generally have a moderately saturated greenish or reddish-yellow hue in their raw forms, and get progressively lighter at higher levels of processing. As the eighteen visual Gardner standards increase from a value of 1 to 18, color goes from light to dark, increasing in dominant yellow saturation while and shifting from a greenish tint to a reddish tint. Gardner standards are well entrenched in numerous industry and manufacturer specifications.

Phytosterol ester products produced in accordance with certain aspects of the present invention typically have Gardner color values of less than about 6, more typically less than about 4 and can be less than about 2 on the Gardner color scale.

Phytosterol esters according to the invention can be added, for example, to food products containing plant oil, such as mustards, salad dressings, and peanut butter. Citric and/or tartaric acid, or their salts, are often added to the aforementioned food products. The unhealthy effects of a meal that is high in fat and cholesterol can be counteracted with the use of these products.

When used in conjunction with a conventional alkoxide transesterification catalyst like sodium methoxide, sodium borohydride should be added prior to the alkoxide for the desired reductive bleaching action to occur. If the sodium borohydride is added after the methoxide, the bleaching action is greatly reduced or even absent. The bleaching action of sodium borohydride is not instantaneous, so a 20-minute stir-out is advisable prior to the subsequent alkoxide catalyst addition. The efficacious amount of sodium borohydride typically ranges from about 0.005% to about 0.1%, more particularly from about 0.01% to about 0.06% and preferably from about 0.02% to 0.04% of the mass of the sterol reactant.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification are approximations that may vary depending upon the desired properties sought to be obtained by the present invention. The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values.

Unless otherwise indicated, all percentages of compositions referred to herein are weight percentages of the total composition (i.e. the sum of all components present) and all ratios are weight ratios.

The invention is described in more detail in the following non-limiting examples:

### EXAMPLE 1

740 grams of fatty acid methyl esters derived from canola and 1000 grams of phytosterol prills were charged to a 3-liter three-necked flask equipped with a thermal well and thermometer, a paddle stirrer, a heating mantle, a “cold finger” (dry ice+acetone) condenser, a receiver of modest capacity (500 ml or less), and a connection to vacuum pump with a manometer, control valve, and trap cooled with dry ice.

This proportion of reactants corresponds to a 6% molar excess of fatty acid methyl esters versus phytosterols. The resulting mixture was heated to 120° C. with vigorous agitation while gradually reducing the pressure to 5 mm Hg or less to remove any water entrained in the phytosterols. If present, moisture will consume the catalyst. The principal (if not the sole) source of moisture is the water contained in the phytosterol prills, which is typically 1% by weight.

Vacuum was broken with nitrogen and 2 grams of the commercial solution of 12% by weight sodium borohydride in 14M aqueous sodium hydroxide was added while agitating vigorously to ensure rapid dispersion. Pressure was gradually reduced to 5 mm Hg and the mixture was allowed to stir out for 20 minutes. During this interval of time the color of the reaction mixture was observed to lighten dramatically.

Vacuum was broken with nitrogen and 4 grams of a 25% by weight solution of sodium methoxide in methanol was added while maintaining vigorous agitation. No darkening of the reaction mixture was observed following the addition of the sodium methoxide solution, which invariably imparts color when borohydride is not employed.

The methanol contained in the catalyst solution flashed off rapidly, followed by the slow, steady evolution of the methanol evolved as the transesterification reaction proceeded. The progress of the reaction was monitored by observing the rate of the evolution of bubbles in reaction mixture and the amount of liquid collected in the receiver. In addition to the methanol and water added as catalyst solution, the transesterification reaction produces an additional 93 milliliters of methanol.

When the rate of reaction appeared to subside, vacuum was broken with nitrogen and another two gram aliquot of catalyst was added. The reaction was allowed to proceed until the amount of unreacted free phytosterols was less than 5.5%, as determined by HPLC analysis of a sample of the reaction mixture and methyl esters were less than 5%, as determined by GC-FID analysis of a sample of the reaction mixture. The total reaction time was 110 minutes.

When the reaction was deemed to be complete, the reaction mixture was washed to remove residual catalyst, spent catalyst, and alkali. After cooling to 95° C., a solution of 8 grams of glacial acetic acid in 250 ml of water was added, followed by vigorous agitation for 15 minutes. The mixture was transferred to a separatory funnel and allowed to stand for 20 minutes. The bottom aqueous layer was removed and discarded.

The supernatant organic layer was charged back to flask. 150 ml of water was added for a second wash. The mixture was heated to 95° C. and agitated vigorously for 15 minutes, transferred to a separatory funnel, and allowed to stand for 20 minutes. The bottom aqueous was removed and discarded, and the supernatant organic layer was transferred to a clean flask. Entrained water was removed at a pressure of 10 mm Hg while gradually heating to 135° C. The product was cooled, blanketed with nitrogen, and dispensed into a sealed bottle. This material measured 2.8 on the Gardner Scale, compared to a value of 5.6 for control material made from the same reactants catalyzed with an identical amount of a methanoic solution of sodium methoxide.

Sodium borohydride is also capable of serving as the sole catalyst for the transesterification reaction, although it is necessary to increase the amount employed by a factor of ten compared to when it is used in conjunction with a conventional alkoxide catalyst, corresponding to approximately 0.2% to 0.4% sodium borohydride relative to the mass of the sterol reactant. However, even with the increased amount of borohydride, the rate of reaction is appreciably slower than when sodium methoxide is employed as the principal catalyst, as shown in the following example.

### EXAMPLE 2

740 grams of fatty acid methyl esters derived from canola and 1000 grams of phytosterol prills were charged to a 3-liter three-necked flask equipped with a thermal well and thermometer, a paddle stirrer, a heating mantle, a “cold finger” (dry ice+acetone) condenser, a receiver of modest capacity (500 ml or less), and a connection to vacuum pump with a manometer, control valve, and trap cooled with dry ice.

This proportion of reactants corresponds to a 6% molar excess of fatty acid methyl esters versus phytosterols. The resulting mixture was heated to 135° C. with vigorous agitation while gradually reducing the pressure to 5 mm Hg or less to remove any water entrained in the phytosterols. When present, moisture will consume the catalyst. The principal (if not the sole) source of moisture is the water contained in the phytosterol prills, which is typically 1% by weight.

The catalyst was prepared by slowly adding 20 grams of commercial aqueous 12% sodium borohydride +40% sodium hydroxide solution to 20 grams of methanol with stirring. The catalytic activity of the resulting solution does not diminish appreciably during a 24-hour interval.

Vacuum was broken and 13 grams of the catalyst solution were added while agitating vigorously to ensure rapid dispersion. Pressure was gradually reduced to 5 mm Hg.

The methanol and water contained in the catalyst solution flashed off rapidly, followed by the slow, steady evolution of the methanol evolved as the transesterification reaction proceeded. The progress of the reaction was monitored by observing the rate of the evolution of bubbles in reaction mixture and the amount of liquid collected in the receiver. In addition to the methanol and water added as catalyst solution, the transesterification reaction produces an additional 93 milliliters of methanol.

When the rate of reaction appeared to subside, vacuum was broken with nitrogen and another two gram aliquot of catalyst was added. The reaction was allowed to proceed until the amount of unreacted free phytosterols was less than 5.5%, as determined by HPLC analysis of a sample of the reaction mixture and methyl esters were less than 5%, as determined by GC-FID analysis of a sample of the reaction mixture. The total reaction time was 260 minutes.

When the reaction was deemed to be complete, the reaction mixture was washed to remove residual catalyst, spent catalyst, and alkali. After cooling to 95° C., a solution of 8 grams of glacial acetic acid in 250 ml of water was added, followed by vigorous agitation for 15 minutes. The mixture was transferred to a separatory funnel and allowed to stand for 20 minutes. The bottom aqueous layer was removed and discarded.

The supernatant organic layer was charged back to flask. 150 ml of water was added for a second wash. The mixture was heated to 95° C. and agitated vigorously for 15 minutes, transferred to a separatory funnel, and allowed to stand for 20 minutes. The bottom aqueous was removed and discarded, and the supernatant organic layer was transferred to a clean flask. Entrained water was removed at a pressure of 10 mm Hg while gradually heating to 135° C. The product was cooled, blanketed with nitrogen, and dispensed into a sealed bottle. This material measured 2.5 on the Gardner Scale, compared to a value of 5.6 for control material made from the same reactants catalyzed with an identical amount of a methanoic solution of sodium methoxide.

What is claimed is:

1. A method for the synthesis of phytosterol esters comprising:
providing a mixture comprising free sterols and fatty acid esters of low molecular weight aliphatic alcohols; contacting said mixture with a reductive bleaching agent; and
reacting the sterols and fatty acid esters in the presence of sufficient catalyst to produce the corresponding phytosterol esters.

2. The method of claim 1 wherein said reductive bleaching agent is an alkali metal borohydride selected from the group consisting of sodium borohydride, potassium borohydride and combinations thereof.

3. The method of claim 1 wherein said catalyst comprises an alkali metal borohydride selected from the group consisting of sodium borohydride, potassium borohydride and combinations thereof.

4. The method of claim 1 wherein said catalyst comprises an alkali metal alkoxide.

5. The method of claim 4 wherein said reductive bleaching agent is contacted with the mixture prior to the addition of any alkali metal alkoxide catalyst.

6. The method of claim 5 further comprising stirring-out said mixture and said reductive bleaching agent for an interval of between about five minutes and about one hour prior to the addition of the alkoxide catalyst.

7. The method of claim 1 further comprising washing out the phytosterol product with a dilute solution of a weak, volatile organic acid to wash out residual borohydride, borates, and sodium soaps of fatty acids after the completion of the transesterification reaction.
